(19) United States
(12) Patent Application Publication
(10) Pub. No.: US 2022/0111092 A1
(43) Pub. Date: Apr. 14, 2022

(54) DEVICE FOR THE GENERATION OF OZONE

(71) Applicant: SALL S.R.L., Cavriago (IT)

(72) Inventor: Marco Luigi Tassi, Montù Beccaria (PV) (IT)

(73) Assignee: SALL S.R.L., Cavriago (IT)

(21) Appl. No.: 17/398,784

(22) Filed: Aug. 10, 2021

(30) Foreign Application Priority Data
Oct. 13, 2020 (IT) 102020000024058

(51) Int. Cl. A61L 2/20; B01D 46/00; B01D 53/02; C01B 13/11

(52) U.S. Cl. CPC A61L 2/202; C01B 2201/62; B01D 46/003; B01D 53/02; C01B 13/11; B01D 2253/108; B01D 2253/102; B01D 2253/104; B01D 2257/304; B01D 2257/504; B01D 2257/80; B01D 2257/406; B01D 2257/7022; A61L 2202/11; B01D 46/0023

(57) ABSTRACT

A device for the generation of ozone, comprising: at least one ozone-producing cell equipped with an inlet and with an outlet and adapted to convert into ozone at least a part of the oxygen contained in an aeriform stream of environment air that passes through it from said inlet towards said outlet, at least one conveying duct configured to convey environment air towards the inlet of the ozone-producing cell, a filtering arrangement placed along said conveying duct to filter the environment air before it reaches the ozone-producing cell, wherein said filtering arrangement comprises a granular filter comprising at least silicate granules.

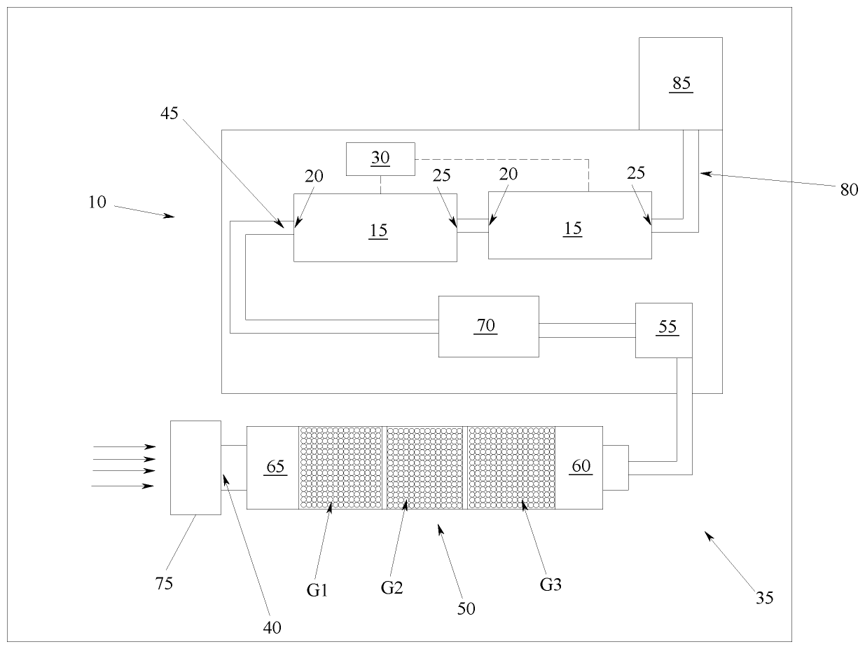

### CROSS-REFERENCE TO RELATED APPLICATION

This patent application claims the benefit of priority to Italian Patent Application No. 102020000024058, filed Oct. 13, 2020, and entitled, “Device for the Generation of Ozone,” which is incorporated in its entirety herein by this reference.

### TECHNICAL FIELD

The present disclosure relates to the field of devices for the decontamination of environments from pathogens such as viruses, bacteria and spores, and unwanted chemical compounds. In particular, the disclosure relates to a device for the generation of ozone for the decontamination of an environment.

### BACKGROUND

In the field of the treatments for decontaminating environments, the use of ozonizers, i.e., devices capable of generating ozone starting from oxygen, is known. Some ozonizers are configured in such a way as to suck air from the environment and to generate, starting from the oxygen present in the sucked air, ozone which is subsequently introduced into the environment to be sanitized.

In order to generate ozone, the air is made to pass through two frames, one of which is positively charged and the other one is negatively charged. By applying a suitable voltage to said frames, the so-called corona effect is generated which allows the bonds between the atoms of oxygen present in the air to be modified to promote the formation of ozone. A drawback known in the art is that the air passing through the frames must be suitably filtered. In fact, the possible presence of impurities and/or humidity could lead to the formation of electrical discharges between the frames and thus to the damage of the ozonizer and/or to the formation of γ (gamma) rays which, as known, are highly harmful to human beings.

It will be appreciated that this background description has been provided to aid the reader, and is not to be taken as an indication that any of the indicated problems were themselves appreciated in the art. While the described principles can, in some aspects and embodiments, alleviate the problems inherent in other systems, it will be appreciated that the scope of the protected innovation is defined by the attached claims, and not by the ability of any disclosed feature to solve any specific problem noted herein.

### SUMMARY

The present disclosure, in one aspect, is directed to embodiments of a device for the generation of ozone. In embodiments, a device for the generation of ozone includes an ozone-producing cell, a conveying duct, and a filtering arrangement. The ozone-producing cell has an inlet and an outlet and is adapted to convert into ozone at least a part of an amount of oxygen contained in a flow of gas entering in the inlet, passing through the ozone-producing cell, and discharging out the outlet. The conveying duct is in fluid communication with the inlet of the ozone-producing cell and is configured to convey the flow of gas to the inlet of the ozone-producing cell. The filtering arrangement is arranged with the conveying duct to filter the flow of gas upstream of the inlet of the ozone-producing cell. The filtering arrangement includes a granular filter comprising silicate granules.

Further and alternative aspects and features of the disclosed principles will be appreciated from the following detailed description and the accompanying drawings. As will be appreciated, features of a device for the generation of ozone disclosed herein are capable of being carried out in other and different embodiments, and capable of being modified in various respects. Accordingly, it is to be understood that both the foregoing general description and the following detailed description are exemplary and explanatory only and do not restrict the scope of the appended claims.

### BRIEF DESCRIPTION OF THE DRAWINGS

Further features and advantages of the invention will be more apparent after reading the following description provided by way of a non-limiting example, with the aid of the figure.

The FIGURE is a schematic view of an exemplary embodiment of a device for the generation of ozone according to the invention.

### DETAILED DESCRIPTION OF EXEMPLARY EMBODIMENTS

The present disclosure is directed to embodiments of a device for the generation of ozone. In embodiments, a device for the generation of ozone constructed according to principles of the present disclosure includes an ozone-producing cell, a conveying duct, and a filtering arrangement. The ozone-producing cell (preferably with corona effect) has an inlet and an outlet and is adapted to convert into ozone at least a part of an amount of oxygen contained in a flow of gas entering in the inlet, passing through the ozone-producing cell, and discharging out the outlet. The conveying duct is in fluid communication with the inlet of the ozone-producing cell and is configured to convey the flow of gas toward the inlet of the ozone-producing cell. The filtering arrangement is arranged with the conveying duct to filter the flow of gas upstream of the inlet of the ozone-producing cell. The filtering arrangement includes a granular filter comprising silicate granules.

In embodiments of the invention, a device for the generation of ozone constructed according to principles of the present disclosure is equipped with a granular filter that allows to effectively absorb the humidity present in the environment air by means of the aforesaid silicate granules, obviating the possibility that electric discharges and/or γ (gamma) rays might occur.

In embodiments of the invention, a device for the generation of ozone constructed according to principles of the present disclosure includes a granular filter comprising brown type silicate granules and/or white type silicate granules. In at least some of such embodiments, the granular filter can exploit the combined action of brown and white type silicates.

In particular, the brown type silicate can act as an indicator of consumption of the granular filter, in order to signal the necessary replacement. In fact, this brown type silicate shows a characteristic coloring that varies according to the state of consumption thereof, i.e. it changes color (for example it turns from brown to blue) as the silicate absorbs humidity.

In embodiments of the invention, the silicate granules can have a maximum size (e.g. diameter) comprised between 0.5 mm and 6 mm, preferably comprised between 2 mm and 4 mm, even more preferably comprised between 0.5 mm and 2 mm. In at least some of such embodiments, the silicate granules have a size that allows creating a dense barrier for the environment air, increasing the contact surface and therefore the absorbing capacity.

In embodiments of the invention, the quantity of silicate granules can be such as to occupy globally a volume higher than 50% of the total volume occupied by all the granules that make up the granular filter. In at least some of such embodiments, the effect of absorbing the humidity present in the environment air is particularly effective, i.e. the granular filter is able to absorb a high quantity of humidity before having to be replaced, due to the large volume occupied by the silicate granules.

In embodiments of the invention, the granular filter can further comprise zeolite granules. In at least some of such embodiments, these zeolite granules act as a molecular sieve, and the granular filter is particularly effective in retaining hydrocarbons and/or ammonia compounds that may be present in the environment air that bind to the zeolite remaining trapped, thus obviating the possibility that gamma rays might form.

In embodiments of the invention, the granular filter can further comprise zeolite granules having a maximum size (e.g. diameter) comprised between 0.5 mm and 3 mm, for example comprised between 2 mm and 3 mm, but preferably comprised between 0.5 mm and 2.5 mm. In at least some of such embodiments, the zeolite granules have a size that allows creating a dense filtering barrier for the environment air, which slows down the flow of environment air and makes the retention of hydrocarbons and/or other compounds more effective, improving further the filtering action.

In embodiments of the invention, the granular filter can further comprise zeolite granules having porosities (i.e. pore sizes) lower than 10 Angstroms, preferably about 4 Angstroms. In at least some of such embodiments, the zeolite granules are even more effective in retaining some respective contaminants, particularly of hydrocarbons and of ammonia compounds, since the smaller the pore size, the greater the active surface of the zeolite granules with respect to the occupied volume.

In embodiments of the invention, the granular filter can further comprise zeolite granules with a quantity of zeolite granules such as to occupy globally a higher volume comprised between 18% and 35% of the total volume occupied by all the granules that make up the granular filter. In at least some of such embodiments, due to the large volume occupied by the zeolite granules, i.e. to the high quantity thereof, the granular filter is able to retain a high quantity of hydrocarbons and/or ammonia compounds, before having to be replaced.

In embodiments of the invention, the granular filter can further comprise activated carbon granules. In at least some of such embodiments, the granular filter is particularly effective in filtering/retaining a multiplicity of other contaminating substances that may be present in the environment air, such as nitrates.

In embodiments of the invention, the activated carbon granules can have a porosity (i.e. pore size) comprised between 400 and 600 Angstroms (about 500 Angstroms). In at least some of such embodiments, due to this reduced size of the pores of the activated carbon granules, the filtering action of the corresponding contaminants is particularly effective, by virtue of the greater ratio between active surface and volume of the granules.

In embodiments of the invention, the granular filter may comprise activated alumina. In at least some of such embodiments, the granular filter is equipped with granules which are particularly effective in retaining humidity and/or other types of contaminating substances that may be present in the environment air. Furthermore, compared to silicates, the activated alumina granules have the advantage that they can be regenerated.

In embodiments of the invention, the silicate granules, the zeolite granules, and the activated carbon granules can occupy separate volumes arranged in succession along the conveying duct. In at least some of such embodiments, the granular filter allows to carry out various filtration stages in succession, each of which is particularly effective in retaining particular types of contaminating substances.

Still, a further aspect of embodiments of the invention provides that the filtering arrangement can comprise a first mechanical filter arranged along the conveying duct between the granular filter and the ozone-producing cell to retain particles with sizes greater than 0.25 microns. In at least some of such embodiments, the first mechanical filter performs a fine filtration that allows to remove solid particles even of extremely small size from the environment air, preventing them from reaching the ozone-producing cell, where otherwise they might settle and give rise to unwanted phenomena like electric discharges.

Another aspect of embodiments of the invention provides that said first mechanical filter can be hydrophobic. In at least some of such embodiments, when the environment air passes through the first mechanical filter, the water molecules that have not been completely absorbed in the granular filter are effectively rejected, thus obviating the possibility of subsequent generation of electrical discharges due to the presence of humidity in the environment air.

Still another aspect of embodiments of the invention provides that the filtering arrangement can comprise a second mechanical filter arranged along the conveying duct between the granular filter and the first mechanical filter to retain particles with a size greater than 2 microns. In at least some of such embodiments, the second mechanical filter performs a pre-filtration stage, upstream of the fine filtration stage performed by the first mechanical filter, which allows to block solid particles with relatively larger sizes, protecting the first mechanical filter and therefore avoiding that it can clog up very quickly.

A further aspect of embodiments of the invention provides that the filtering arrangement can comprise a third mechanical filter placed along the conveying duct upstream of the granular filter with respect to the conveying direction of the environment air, which is configured to retain particles with sizes greater than 10 micron. In at least some of such embodiments, the third mechanical filter performs a preliminary filtration stage which allows to retain the coarser solid particles, preventing them from obstructing the granular filter and eventually reaching the mechanical filters placed downstream, increasing the useful life thereof.

Still, a further aspect of embodiments of the invention provides that the device can comprise a suction unit configured to suck a quantity of environment air from the outside and to introduce it into the conveying duct. In at least some of such embodiments, the device is configured to create the forced flow of environment air that passes through the filtering group and the ozone-producing cell from the inlet towards the outlet.

Another aspect of embodiments of the invention provides that the suction unit can be placed along the conveying duct between the filtering arrangement and the ozone-producing cell. In at least some of such embodiments, the filtering arrangement works under depression, so that the air flow that passes through it moves at a reduced speed which allows improving the filtration effect.

Still another aspect of embodiments of the invention provides that the device can comprise a non-return valve associated with an inlet mouth of the conveying duct upstream of the filtering arrangement with respect to the conveying direction of the environment air. In at least some of such embodiments, the device is configured in such a way as to preserve the integrity of the filtering arrangement, and in particular of the granules that make up the granular filter, substantially by selectively closing the conveying duct when the device for the generation of ozone does is not activated.

A further aspect of embodiments of the invention provides that the device can comprise two ozone-producing cells arranged in series one to another. In at least some of such embodiments, the device is configured in such a way as to be able to generate a greater quantity of ozone in the unit of time, in fact the oxygen present in the environment air not transformed into ozone at the first ozone-producing cell is reprocessed at the second ozone-producing cell in order to produce additional ozone.

With particular reference to the aforementioned FIGURE, the reference number 10 globally indicates a device for the generation of ozone. The device 10 is adapted to be arranged inside an environment A to be sanitized, particularly to sanitize the air of said environment A and of all the surfaces present inside. The environment A is preferably a closed or partially closed or circumscribed environment.

It is specified that decontamination means an operation aimed at the reduction by removing and/or killing and/or inactivating bacteria, viruses, fungi, protozoa, spores, and at the elimination of unwanted chemical compounds in order to control/cancel the risk of infection for people or of contamination of objects or environments.

The device 10 first of all comprises at least one ozone-producing cell 15, i.e. a cell adapted to convert into ozone at least a part of the oxygen contained in a flow of gas that passes through it. In the embodiment described herein, the gas stream is a flow of environment air, for example a quantity of air withdrawn from the environment A in which the device 10 is located or any other external environment.

More in detail, the ozone-producing cell 15 is substantially constituted by a central electrode, preferably made of stainless steel, and by an external tube made of dielectric material that surrounds the central electrode, preferably made of Pyrex (commercially-available from Corning Inc. of New York), between which an interspace remains defined. The ozone-producing cell 15 also has an inlet 20 and an outlet 25, which are connected by means of the interspace. In this way, the aeriform stream of environment air which moves from the inlet 20 towards the outlet 25 is forced to pass through the interspace defined between the central electrode and the external tube.

The central electrode and the external tube of the ozone-producing cell 15 are adapted to be connected the former to a suitable power supply voltage, for example a nominal power supply voltage comprised between 3000 and 8000 Vac and at a frequency comprised between 18 and 30 KHz, preferably equal to 20 KHz, the latter to a reference potential, for example to the ground. In this way, a so-called corona effect is created at the interspace defined between the central electrode and the external tube, which allows the generation of ozone starting from the oxygen present in the aeriform stream that passes through the ozone-producing cell 15.

For the above purpose, the device 10 can comprise a voltage drive circuit 30 which, once it is connected to a suitable power supply source (for example to the electrical network), allows the ozone-producing cell 15 to be supplied with the aforesaid suitable power supply voltage and at the aforesaid suitable frequency.

In the illustrated example, the device 10 comprises in particular a series of ozone-producing cells 15, i.e. at least two ozone-producing cell cells 15 connected in series one to another. In other words, the outlet 25 of the first ozone-producing cell 15 is sealingly connected to the inlet 20 of the second ozone-producing cell 15, so that said second ozone-producing cell 15 can be crossed by the aeriform stream leaving the first ozone-producing cell 15. However, it is not excluded that, in other embodiments, the ozone-producing cells 15 of the device 10 can be mutually connected in parallel rather than in series.

The device 10 further comprises a conveying duct 35, which is configured to convey a flow of environment air to at least one ozone-producing cell 15, for example to the first ozone-producing cell 15 of the series. The conveying duct 35 has an inlet mouth 40, in fluidic connection (direct or indirect) with the environment A or with any other external environment, and an opposing outlet mouth 45, which is sealingly connected to the inlet 20 of the ozone-producing cell 15. In this way, the conveying duct 35 is adapted to convey a quantity of environment air in the unit of time (i.e. an air flow) which moves in a predetermined conveying direction from the inlet mouth 40 to the outlet mouth 45.

The device 10 comprises a filtering arrangement placed along the conveying duct 35, i.e. interposed between the inlet mouth 40 and the outlet mouth 45, and which itself defines a section of said conveying duct 35. In other words, the inlet mouth 40 and the outlet mouth 45 of the conveying duct 35 are in fluidic communication exclusively/solely through the filtering arrangement. The filtering arrangement is configured to filter the environment air before it reaches the ozone-producing cell 15, so as to retain humidity and contaminating substances present therein which might otherwise cause electrical discharges and/or the formation of gamma rays.

The filtering arrangement first of all comprises a granular filter 50, i.e. a filter consisting of solid bodies of small sizes (granules) which, being heaped together, define a barrier characterized by narrow interstices (present between the various granules). These interstices allow the passage of the airflow but, at the same time, allow the materials constituting the granules to retain any impurities and/or humidity and/or other chemical substances that might be harmful for the correct operation of the ozone-producing cell 15.

For this purpose, the granular filter 50 can comprise silicate G1 granules, which have the main function of retaining humidity. The silicate G1 granules can have a maximum size (for example diameter) comprised between 0.5 mm and 6 mm, preferably comprised between 2 mm and 4 mm, even more preferably comprised between 0.5 mm and 2 mm. Preferably, the quantity of silicate G1 granules is such as to occupy globally a volume higher than 50% of the total volume occupied by all the granules that make up the granular filter 50.

In the particular case in which the environment A to be decontaminated is a purely home environment, the quantity of silicate G1 granules can constitute the entire quantity of granules that make up the granular filter 50. More specifically, the granular filter 50 can comprise brown type silicate G1 granules and/or white type silicate granules.

“White type” silicate means in the present discussion a dehydrating silica gel (SiO2), obtained by synthesis, which is presented in the form of granules characterized by a white/optically transparent coloring. This white type silicate can correspond to CAS number 112926-00-8, where the CAS number is the numerical identifier that uniquely identifies a chemical substance assigned by the Chemical Abstracts Service.

“Brown type” silicate means in the present discussion a dehydrating silica gel (SiO2), obtained by synthesis, which is presented in the form of granules characterized by a brown coloring. This coloring, in particular, can turn from brown to light blue when the silicate has reached saturation, i.e. it has exhausted its ability to adsorb. This brown type silicate can correspond to CAS number 7631-86-9, where the CAS number is the numerical identifier that uniquely identifies a chemical substance assigned by the Chemical Abstracts Service.

Preferably, the granular filter 50 comprises both brown type silicate granules and white type silicate granules, which can be present, for example, in equal quantities and can be mixed together in the most uniform way possible.

The granular filter 50 can also comprise zeolite G2 granules, which are particularly effective in retaining ammonia compounds and/or hydrocarbons possibly present in the environment air, binding to them chemically. These zeolite G2 granules are, in particular, able to retain various types of polar molecules including water, ammonia, methanol, H2S, CO2, CH3OH, C3H4, C2H6, C3H6.

Zeolite refers to a synthetic dehydrating material containing sodium, aluminum, silicon and oxygen. The zeolite G2 granules preferably have a maximum size (intended for example as the diameter) comprised between 0.5 mm and 3 mm, for example comprised between 2 mm and 3 mm, preferably comprised between 0.5 mm and 2.5 mm.

Furthermore, the quantity of zeolite G2 granules can be such as to occupy globally a volume comprised between 18% and 35% of the total volume occupied by all the granules that make up the granular filter 50. For example, particularly in the case in which the environment A to be decontaminated is an environment for office use, the granules of the granular filter 50 can consist of ⅔ of silicate G1 granules and of ⅓ of zeolite G2 granules.

The zeolite G2 granules of the granular filter 50 are generally porous and preferably have a porosity (i.e. pore size) lower than 10 Angstroms, for example substantially equal to about 4 Angstroms.

The granular filter 50 can further comprise activated carbon G3 granules, particularly effective in chemically binding with, and therefore retaining, numerous other substances that may be present in the environment air, including in particular nitrate compounds. Preferably, the activated carbon G3 granules can have the characteristics shown in the following table:

| Grain size | 30 × 8 mesh (0.6-2.4 mm) | |
|---|---|---|
| Specific surface | 900 | m²/g |
| Ash content | 5% | |
| Apparent density | 0.50 | g/cm³ |
| Humidity | 3% | max |
| Iodine value | 800 | mg/g |
| Methylene blue | 200 | mg/g |

| | |
|---|---|
| Effective particle size | >8 mesh max 5% − <30 mesh max 5% |

Preferably, the activated carbon granules can have a maximum size (intended for example as the diameter) comprised between 0.6 mm and 6 mm, for example comprised between 2 mm and 6 mm, but preferably comprised between 0.6 mm and 2.4 mm. Furthermore, the activated carbon granules of the granular filter 50 are porous and preferably have a porosity comprised between 400 and 600 Angstroms, for example about 500 Angstroms.

The quantity of activated carbon granules can be such as to occupy globally a volume comprised between 15% and 25% of the total volume occupied by all the granules that make up the granular filter 50, for example about 20% of said total volume. For example, particularly in the case in which the environment A to be decontaminated is a particularly critical environment, such as for example a seasoning cell, the granules of the granular filter 50 can consist of ⅗ of silicate G1 granules, of ⅕ of zeolite G2 granules and of ⅕ of activated carbon G3 granules.

Alternatively or in addition, the granular filter 50 can comprise activated alumina granules, which are capable of binding with, and therefore retaining, humidity and a wide variety of different compounds that may possibly be present in the environment air.

The different types of granules of the granular filter 50, i.e. the silicate G1 granules (white type and/or brown type) and/or the zeolite G2 granules and/or activated carbon G3 granules and/or activated alumina granules, can be mixed together or they can occupy separate volumes arranged in succession along the conveying duct 35. For example, the granules of the various types can be housed in a single containment casing connected to the conveying duct 35, and be separated by type, for example by means of a filter paper disc. Alternatively, it is possible to provide that the granules of each type can each be housed in a respective container casing connected to the conveying duct 35, and that said container casings are connected in series sealingly one to another, so that the flow of environment air outflowing from one of said container casings enters the subsequent one.

In addition to the granular filter 50, the filtering arrangement can comprise one or more mechanical filters adapted to retain the solid particles. In particular, the filtering arrangement can comprise a first mechanical filter 55, which is arranged along the conveying duct 35 between the granular filter 50 and the ozone-producing cell 15, i.e. downstream of the granular filter 50 and upstream of the ozone-producing cell 15 with respect to the conveying direction of the environment air from the inlet mouth 40 to outlet mouth 45.

The first mechanical filter 55, which can be a class 2A mechanical filter according to the UNI10339 standard, is preferably configured to retain particles with sizes greater than or equal to 0.25 microns. Furthermore, the first mechanical filter 55 is preferably hydrophobic, so as to be able to prevent the passage towards the ozone-producing cell 15 of any humidity that may have escaped from the granular filter 50.

The filtering arrangement can additionally comprise a second mechanical filter 60 arranged along the conveying duct 35 between the granular filter 50 and the first mechanical filter 55, i.e. downstream of the granular filter 50 and upstream of the first mechanical filter 55 with respect to the conveying direction of the environment air from the inlet mouth 40 to the outlet mouth 45. The second mechanical filter 60, which can be constituted by a layer of porous foam, is preferably configured to retain particles with a size greater than or equal to 2 microns.

The filtering arrangement may also comprise a third mechanical filter 65 placed along the conveying duct 35 upstream of the granular filter 50 with respect to the conveying direction of the environment air. The third mechanical filter 65 can be configured to retain particles with sizes greater than 10 microns. For example, the third mechanical filter 65 can also be constituted by a layer of porous foam.

In order to generate the flow of air which runs through the conveying duct from the inlet mouth 40 towards the outlet mouth 45, the device 10 can comprise a suction unit 70. The suction unit 70 is a device of the known type and therefore not described in detail.

For example, the suction unit 70 can comprise a suction fan or impeller and a drive motor, for example an electric motor, adapted to set said fan or impeller in rotation so as to draw a certain quantity of environment air in the unit of time. The suction unit 70 can be connected sealingly to the conveying duct 35.

In particular, the suction unit 70 can be located along the conveying duct 35 between the filtering arrangement and the ozone-producing cell 15, i.e. downstream of the filtering arrangement and upstream of the ozone-producing cell 15 with respect to the conveying direction of the environment air. In other words, the filtering arrangement can be in fluidic communication with the ozone-producing cell 15 exclusively/solely through the suction unit 70, and the suction unit 70 itself makes available a respective section of the conveying duct 35. Thanks to this arrangement, the suction unit 70 is able to place the filtering arrangement under depression, which is therefore crossed by an airflow that proceeds more slowly and more uniformly, making it possible to obtain a particularly effective filtering action.

The device 10 may further comprise a non-return valve 75 associated with the inlet mouth 40 of the conveying duct 35. The non-return valve 75 allows to regulate, on the basis of a pressure differential that is created between the inside of the conveying duct 35 and the outside, the opening and closing of the inlet mouth 40 for the inlet 20 of the environment air. The non-return valve 75 allows, in other words, to close the inlet mouth 40 of the conveying duct 35 when the device 10 is not in operation, i.e. when the suction unit 70 is off, so as to prevent that environment air can continue to enter and consume/degrade the granules of the granular filter 50.

In practice, when the suction unit 70 is activated, for example when the motor that drives the fan or impeller is driven, a depression is generated which allows the opening of the non-return valve 75 and therefore the inlet 20 of environment air flow inside the conveying duct 35. On the other hand, when the motor that drives the fan or impeller is off, the non-return valve 75 is closed.

Finally, the device 10 can be completed by a delivery duct 80, which is sealingly connected to the outlet 25 of the ozone-producing cell 15, for example of the second ozone-producing cell 15 of the series, and is freely open towards the environment A. In this way, the delivery duct 80 is capable of delivering into the environment A the ozone produced in the ozone-producing cell 15 and what remains of the flow of environment air.

A diffuser 85 can be associated with the delivery duct 80, which is configured to diffuse (i.e. to project to the outside) the ozone generated by the ozone-producing cell 15. For example, the diffuser 85 may comprise a fan or impeller the rotation of which is driven by a suitable drive motor.

The invention thus conceived is susceptible to several modifications and variations, all falling within the scope of the inventive concept. Moreover, all details can be replaced by other technically equivalent elements. In practice, the materials used, as well as the contingent shapes and sizes, can be whatever according to the requirements without for this reason departing from the scope of protection of the following claims.

What is claimed is:

1. A device for the generation of ozone, comprising:
an ozone-producing cell having an inlet and an outlet and being adapted to convert into ozone at least a part of an amount of oxygen contained in a flow of gas entering in the inlet, passing through the ozone-producing cell, and discharging out the outlet;
a conveying duct in fluid communication with the inlet of the ozone-producing cell and configured to convey the flow of gas to the inlet of the ozone-producing cell;
a filtering arrangement arranged with the conveying duct to filter the flow of gas upstream of the inlet of the ozone-producing cell;
wherein the filtering arrangement includes a granular filter comprising silicate granules.

2. The device according to claim 1, wherein the granular filter comprises at least one of brown type silicate granules and white type silicate granules.

3. The device according to claim 1, wherein the granular filter further comprises zeolite granules.

4. The device according to claim 1, wherein the granular filter further comprises activated carbon granules.

5. The device according to claim 1, wherein the granular filter further comprises activated alumina.

6. The device according to claim 1, wherein the quantity of silicate granules is such as to occupy globally a volume higher than 50% of the total volume occupied by all the granules that make up the granular filter.

7. The device according to claim 6, wherein the granular filter further comprises zeolite granules.

8. The device according to claim 7, wherein the quantity of zeolite granules is such as to occupy globally a higher volume comprised between 18% and 35% of the total volume occupied by all the granules that make up the granular filter.

9. The device according to claim 8, wherein the granular filter further comprises activated carbon granules.

10. The device according to claim 8, wherein the granular filter further comprises activated alumina.

11. The device according to claim 1, wherein the filtering arrangement comprises a first mechanical filter arranged with the conveying duct between the granular filter and the ozone-producing cell and adapted to retain particles with sizes greater than 0.25 microns.

12. The device according to claim 11, wherein said first mechanical filter is hydrophobic.

13. The device according to claim 11, wherein the filtering arrangement comprises a second mechanical filter arranged with the conveying duct between the granular filter and the first mechanical filter and adapted to retain particles with a size greater than 2 microns.

14. The device according to claim 13, wherein the filtering arrangement comprises a third mechanical filter arranged with the conveying duct upstream of the granular filter with respect to the direction of the flow of gas, the third mechanical filter being configured to retain particles with sizes greater than 10 microns.

15. The device according to claim 1, further comprising:
a suction unit configured to suck a quantity of environment air from the outside and to introduce the quantity of environment air into the conveying duct as the flow of gas, said suction unit being located along the conveying duct between the filtering arrangement and the ozone-producing cell.

16. The device according to claim 1, further comprising:
a non-return valve associated with an inlet mouth of the conveying duct upstream of the filtering arrangement with respect to the direction of the flow of gas.
